(19) United States
(12) Patent Application Publication
(10) Pub. No.: US 2008/0305056 A1
(43) Pub. Date: Dec. 11, 2008

(54) STABLE, LOW VISCOSITY COSMETIC COMPOSITIONS

(75) Inventors: Klaus Jenni, Essen (DE); Ralf Mathiak, Gladbeck (DE); Marcel Veeger, Goch (DE); Jurgen Meyer, Munster (DE)

Correspondence Address: SCULLY SCOTT MURPHY & PRESSER, PC, 400 GARDEN CITY PLAZA, SUITE 300, GARDEN CITY, NY, 11530, US

(73) Assignee: EVONIK GOLDSCHMIDT GMBH, Essen (DE)

(21) Appl. No.: 12/132,307

(22) Filed: Jun. 3, 2008

(30) Foreign Application Priority Data
Jun. 8, 2007 (DE) 102007027030.7

(51) Int. Cl. A61K 8/72; A61Q 19/04; A61Q 17/04; A61Q 19/00

(52) U.S. Cl. 424/59; 424/78.03

(57) ABSTRACT

The invention relates to a cosmetic composition for application to the skin, having:
a) 0.05 to 0.5% by weight of a quaternized polysaccharide, 0.05 to 5.0% by weight of a polyethylene glycol derivative or polypropylene glycol derivative and/or 0.05 to 3% by weight of a polymeric quaternized ammonium compound (“polyquaternium”),
b) 10 to 40% by weight of oils and/or oil-like constituents, and
c) 0.2 to 15% by weight of an ester quat and/or of a dialkyl quat.

### FIELD OF THE INVENTION

The present invention relates to cosmetic compositions for application to the skin which comprise ester quats and/or dialkyl quats.

### BACKGROUND OF THE INVENTION

The term “ester quats” is generally understood as meaning quaternized fatty acid alkanolamine esters and salts thereof. These are cationic surfactants which generally have low toxicity and good biodegradability. Ester quats typically have the following general structural formula:

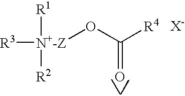

R⁴ is an acyl radical, and Z is an alkylene group. The group Z-OOCR⁴ can also be referred to as a fatty acid alkanol ester group. The radicals R¹ to R³, by contrast, may be hydrogen or alkyl groups or esters of alkylene groups and acyl radicals,
X- is an anion and can, in particular, be chloride, bromide, methosulfate, nitrate, acetate, phosphate or tosylate.

Ester quats have cationic properties in every respect and are therefore classed as cationic surfactants. On account of their particular, conditioning properties, ester quats are typically used in fabric softeners. Additionally, ester quats produce a pleasant hair and skin feel and are therefore used as emulsifiers or conditioners in cosmetic compositions. Moreover, ester quats impart water-resistant properties to such compositions under certain conditions.

Ester quats are substances known per se which can be synthesized by relevant methods of preparative organic chemistry.

The preparation of ester quats is described, for example, in WO-A-91/01295, according to which triethanolamine is partially esterified in the presence of hypophosphorous acid with fatty acids, air is passed through and then quaternization with dimethyl sulfate or ethylene oxide is carried out. Also known from German patent specification DE-C-43 08 794 is a method of producing solid ester quats in which the quaternization of triethanolamine esters is carried out in the presence of suitable fatty alcohols. In a further known method of producing ester quats, it is possible to start from either fatty acids, or the corresponding triglycerides in admixture with dicarboxylic acids. One such production method is described in the European patent specification EP-B-0 750 606. The use of ester quats in cosmetic compositions is known, for example, from DE-A-198 51 451, EP-B-1 239 828, EP-A-1 250 906 and EP-B1 254 653.

Dialkyl quats are likewise positively charged quaternary ammonium compounds which are also used as cationic surfactants and as conditioners. Dialkyl quats are characterized by the fact that the quaternary nitrogen is substituted by at least two alkyl groups each having more than 5 carbon atoms. Dialkyl quats obey the following general structural formula:

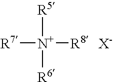

where at least two of the radicals R^{5′} to R^{8′} are alkyl groups. R^{5′} and R^{6′} may, for example, be C₁-₃-alkyl groups, while R^{7′} and R^{8′} may, for example, be C₈-₁₈-alkyl groups. X is an anion and can, in particular, be chloride, bromide, methosulfate, nitrate, acetate, phosphate or tosylate.

It is problematic that ester quats and/or dialkyl quats be present in cosmetic compositions in concentrations which are at least required to achieve the aforementioned effects, particularly when the composition also has oils or oil-like constituents which bring about a high viscosity of the particular cosmetic composition. Furthermore, in this connection, it has been established that low-viscosity formulations, which comprise the specified ester quats and/or dialkyl quats, do not have long-term storage stability without further additives.

Cosmetic compositions with low viscosities have been enjoying, especially recently, ever greater popularity. Such cosmetic compositions can be spread easily and rapidly on the skin and give the consumer the impression that they absorb rapidly into the skin. Added to this is the fact that low-viscosity skincare compositions can be applied to the skin particularly easily using the ever more popular pump sprays and then be spread.

In the case of the cosmetic compositions of the type specified at the start, it is not possible to dispense with oils or oil-like constituents since these have skincare properties. In some cases, oil-like constituents also have other tasks, thus, for example, in the case of sunscreen compositions, which fall under the definition of the cosmetic compositions for application to the skin for the purposes of the present invention, and in which oil-like sunscreen filters are often used.

In view of the above, there is a continued need to provide cosmetic compositions for application to the skin which have low-viscosity properties and long-term storage stability.

### SUMMARY OF THE INVENTION

The present invention provides cosmetic compositions for application to the skin comprising ester quats and/or dialkyl quats which have low-viscosity properties and long-term storage stability.

Accordingly, a cosmetic composition for application to the skin is provided, having:
a) 0.05 to 0.5% by weight of a quaternized polysaccharide, 0.05 to 5.0% by weight of a polyethylene glycol derivative or polypropylene glycol derivative and/or 0.05 to 3% by weight of a polymeric quaternized ammonium compound (“polyquaternium”),
b) 10 to 40% by weight of oils and/or oil-like constituents (oil phase), and
c) 0.2 to 15% by weight of an ester quat and/or of a dialkyl quat.

Besides components a), b) and c), the composition preferably comprises water, preferably enough water to make up the sum of the components to 100% by weight.

### DETAILED DESCRIPTION OF THE INVENTION

As stated above, the present invention provides cosmetic compositions for application to the skin comprising ester quats and/or dialkyl quats which have low-viscosity properties and long-term storage stability. In particular, a cosmetic composition for application to the skin is provided which includes a) 0.05 to 0.5% by weight of a quaternized polysaccharide, 0.05 to 5.0% by weight of a polyethylene glycol derivative or polypropylene glycol derivative and/or 0.05 to 3% by weight of a polymeric quaternized ammonium compound (“polyquaternium”), b) 10 to 40% by weight of oils and/or oil-like constituents (oil phase), and c) 0.2 to 15% by weight of an ester quat and/or of a dialkyl quat. Besides the aforementioned components, the composition of the present invention preferably comprises water, preferably enough water to make up the sum of the components to 100% by weight. The invention will now be described in greater detail. It is noted that the specified range data provided herein are, without exception, to be understood to include the respective limiting values. In addition, the data relating to viscosities refer, without exception, to room temperature.

As is known, both the quaternized polysaccharides to be used, and also the polyethylene glycol derivatives or polypropylene glycol derivatives and the polymeric quaternized ammonium salts act as thickeners in cosmetic compositions.

Thus, for example, the polyethylene glycol distearate supplied by Goldschmidt GmbH under the trade name REWOPAL® PEG 6000 DS is specified as a thickener for shampoos. In the case of polysaccharides, such as, for example, guar or carob seed flour, the thickening effect is sufficiently known, for example, from the food sector.

The background of the invention is that the inventors have established that when using ester quats and/or dialkyl quats as cationic emulsifiers in low-viscosity emulsions, the emulsions generally have inadequate storage stability.

In this connection, the inventors have found for the first time that the addition of quaternized polysaccharides, polyethylene glycol derivatives or polypropylene glycol derivatives or quaternized ammonium compounds in the proportions according to the invention decisively improves the storage stability of such emulsions, without leading to the increase in viscosity that would actually be expected—since the latter are known as thickeners.

Against this background, it is surprising and was unforeseeable by the person skilled in the art that precisely the combination of ester quats and/or dialkyl quats, oils and oil-like constituents and also quaternized polysaccharides, polyethylene glycol derivatives or polypropylene glycol derivatives or quaternized ammonium compounds in the proportions according to the invention leads to a product whose viscosity lies within the very low range for cosmetic compositions claimed in the invention.

The addition of the quaternized polysaccharides, polyethylene glycol derivatives or polypropylene glycol derivatives or quaternized ammonium compounds in proportions according to the invention leads to a diluting effect, although the person skilled in the art would have expected a thickening effect.

In this way, it is therefore possible for the first time to provide a cosmetic composition which comprises the ester quats and/or dialkyl quats that bring about a pleasant skin feel, and also skincare oils and oil-like constituents and, at the same time, has a sufficiently low viscosity to comply with the consumer's wish for a product that can be applied easily and pleasantly.

Preferably, it is provided that the viscosity of the cosmetic composition according to the invention is in the range between 50 and 12 000 mPas. The viscosity is particularly preferably in the range between 50 and 1000 mPas. The preferred viscosity ranges correspond to relatively thin-liquid products. In contrast, standard commercial skin cream has a viscosity of from about 15 000 to 30 000 mPas.

As already described above, the term “ester quats” is generally understood as meaning quaternized alkanolamine fatty acid esters. According to the invention, preference is given to the use of at least one ester quat, with preferred mixtures being those which have the following formula I, formula II and/or formula III:

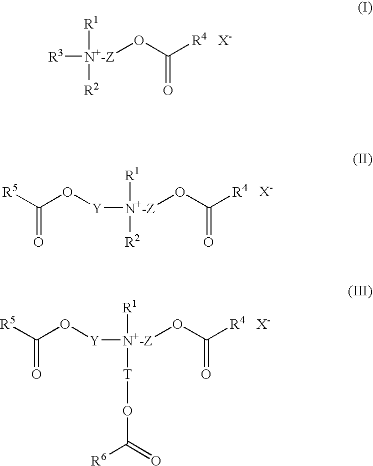

In the formulae (I) to (III), the radicals R¹ to R³ are hydrogen or alkyl groups or esters of alkylene groups and acyl radicals, the radicals R⁴, R⁵ and R⁶ are identical or different and are oleyl, tallow alkyl, stearyl, palmityl, cocoyl, palmyl, rapeseed alkyl, soya alkyl radicals or hydrogenated analogs thereof, and the radicals T, Y and Z are identical or different divalent alkylene radicals, preferably selected from ethylene, propylene, butylene, isopropylene and isobutylene radicals. X is an anion and can, in particular, be chloride, bromide, methosulfate, nitrate, acetate, phosphate or tosylate. Preferably, in the formulae (I) to (III), the radical R¹ is a methyl radical, and the radicals R² and R³ are identical or different and are methyl, hydroxyethyl, hydroxypropyl, hydroxyisopropyl, hydroxybutyl or hydroxyisobutyl radicals.

According to a particularly preferred embodiment of the present invention, it is possible to use ester quats which are selected from:
di(oleylcarboxyethyl)hydroxyethylmethylammonium salt, di(tallow-carboxyethyl)hydroxyethylmethylammonium salt, N,N-di(β-stearoylethyl)-N,N-dimethylammonium salt, N,N-di(β-palmitoylethyl)-N,N-dimethylammonium salt, dicocoylethylhydroxyethylammonium methosulfate, dipalmoylethylhydroxyethylammonium methosulfate, dirapeseed-carboxyethylhydroxyethylammonium methosulfate, and/or disoyacarboxyethylhydroxyethylammonium methosulfate and/or hydrogenated analogs thereof.

According to the present invention, the ester quats can be used individually or in the form of mixtures. With regard to the application properties of the ester quats, for mixtures of ester quats, an average degree of esterification of from 1 to 3, preferably 1.5 to 2.5 and preferably 1.7 to 2.2, has proven particularly advantageous. Furthermore, the use of ester quats which are technical-grade mixtures of mono-, di- and triesters with an average degree of esterification of from 1.5 to 1.9 is preferred. To establish the desired iodine number, the ester quats can be hydrogenated by customary methods.

A particularly preferred ester quat is sold under the trade name REWOQUAT® WE 38 DPG by Goldschmidt. This ester quat is characterized as follows:

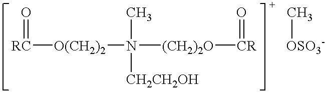

where R is a palmityl radical.

Examples of dialkyl quats that can be used according to the invention are, for example, dioctadecyldimethylammonium chloride (trade name: VARISOFT® TA 100), the structural formula of which is as follows:

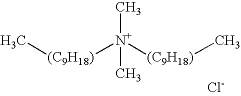

or alkyldihydroxypropyldimethylammonium salts. Further examples include C₁₂₋₁₈-dialkyldimethylammonium chloride, diallyldimethylammonium chloride, dibehenyl/diarachidyldimonium chloride, dibehenyldimonium methosulfate, didecyldimonium chloride, dicetyldimonium chloride (trade name: VARISOFT® 432 PPG), dicetyldimethylammonium chloride, distearyldimethylammonium chloride, dipalmityldimethylammonium chloride, dihydrogentallowdimethylammonium chloride, ditallowdimethylammonium chloride, dihydrogentallowdimethylammonium bromide, or dihydrogentallowdimethylammonium methosulfate, di-C₁₂₋₅₀-alkyldimonium chloride, di-C₁₂₋₁₅-alykyldimonium chloride, di-C₁₄₋₁₈-alkyldimonium chloride, dicapryl/dicaprylyl-dimonium chloride, dicocodimonium chloride, dilaureth-4-dimonium chloride, dilaurylacetyldimethylammoninum chloride, dilauryldimethylammonium chloride, dimer dilinoleamidopropyl-PG-dimethylammonium chloride phosphate, dimethoxycinnamidopropyl-ethyldimethylammonium chloride, ether dimethyl-PABA-ethyl-cetearyldimethylammonium tosylate, dimethyl-PABA-amidopropyl-laurdimonium tosylate, dioleoylisopropyldimethylammonium methosulfate, dipalmitoylethyl-dimethylammonium chloride, dipalmoylisopropyldimethylammonium metho sulfate, disoyadimethylammonium chloride, disteardimethylammonium hectorite, disteareth-6 dimethylammonium chloride, distearoylethyl-dimethylammonium chloride, distearyldimethylammonium chloride, ditallowoyl-PG-dimethylammonium chloride, ditridecyldimethylammonium chloride, sodium borage oil-amidopropyl-PG-dimethylammonium chloride phosphate, sodium cocoamidopropyl-PG-dimethylammonium phosphate, sodium coco-PG-dimethylammonium chloride phosphate, sodium dilinoleamidopropyl-PG-dimethylammonium chloride phosphate, sodium emuamidopropyl-PG-dimethylammonium chloride phosphate, sodium grapeseed oilamidopropyl-PG-dimethylammonium chloride phosphate, sodium hydroxylauryldimonium ethyl phosphate, sodium milkamidopropyl-PG-dimethylammonium chloride phosphate, sodium oleamidopropyl-PG-dimethylammonium chloride phosphate, sodium olive oil-amidopropyl-PG-dimethylammonium chloride phosphate and sodium sunflower oil-amidopropyl-PG-dimethylammonium chloride phosphate.

The oil phase can, for example, be selected from the group of polar oils, comprising lecithins, fatty acid triglycerides, triglycerol esters of saturated and/or unsaturated, branched and/or unbranched alkanecarboxylic acids of chain length from 8 to 24, in particular from 12 to 18, carbon atoms. The fatty acid triglycerides can be selected from the group of synthetic, semisynthetic and/or natural oils, comprising cocoglyceride, olive oil, sunflower oil, soya oil, peanut oil, rapeseed oil, almond oil, palm oil, coconut oil, castor oil, wheatgerm oil, grapeseed oil, thistle oil, evening primrose oil and the like.

Polar oil components can be selected from the group of esters, comprising saturated and/or unsaturated, branched and/or unbranched alkanecarboxylic acids having a chain length from 3 to 30 carbon atoms; saturated and/or unsaturated, branched and/or unbranched alcohols with a chain length from 3 to 30 carbon atoms; esters of aromatic carboxylic acids; saturated and/or unsaturated, branched and/or unbranched alcohols of chain length from 3 to 30 carbon atoms.

Preferred ester oils include octyl palmitate, octyl cocoate, octyl isostearate, octyldodecyl myristate, octyldodecanol, cetearyl isononanoate, isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, stearyl heptanoate, 3-oleyl oleate, oleyl erucate, erucyl oleate, erucyl erucate, tridecyl stearate, tridecyl trimellitate, and also synthetic, semisynthetic and natural mixtures of such esters, in particular jojoba oil.

The oil phase can, however, also comprise dialkyl ethers and dialkyl carbonates, dicaprylyl ethers and/or dicaprylyl carbonate. Suitable oil components can be selected from the group comprising isoeicosane, neopentyl glycol diheptanoate, propylene glycol dicaprylate/dicaprate, caprylic/capric/diglyceryl succinate, butylene glycol dicaprylate/dicaprate, C₁₂₋₁₃-alkyl lactate, di-C₁₂₋₁₃-alkyl tartrate, triisostearin, dipentaerythrityl hexacaprylate/hexacaprate, propylene glycol monoisostearate, tricapryin, dimethyl isosorbide, C₁₂₋₁₅-alkyl benzoate, butyloctyl salicylate, hexadecyl benzoate and butyloctyl benzoate and mixtures thereof.

As an oil phase, it is also possible to use nonpolar oils, including branched and unbranched hydrocarbons and hydrocarbon waxes, in particular mineral oil, vaseline, paraffin oil, squalane and squalene, polyolefins, hydrogenated polyisobutenes and isohexadecane.

The oil phase can also have a content of cyclic or linear silicone oils, or consist entirely of such oils. Systematically, the silicone oils are referred to as polyorganosiloxanes. The methyl-substituted polyorganosiloxanes, which are the most important compounds of this group in terms of amount, are also referred to as polydimethylsiloxane or dimethicones (INCI). Dimethicones come in various chain lengths and with various molecular weights. Suitable polyorganosiloxanes according to the present invention include dimethylpolysiloxanes, which are available, for example, under the trade names ABIL® 10 to 10000 from Goldschmidt GmbH, cetyldimethicones, hexamethylcyclotrisiloxane, polydimethylsiloxane, poly(methylphenylsiloxane), phenylmethylpolysiloxane (INCI: Phenyl Dimethicones, Phenyl Trimethicones), cyclic silicones (octamethylcyclotetrasiloxane or decamethylcyclopentasiloxane), which are also referred to in accordance with INCI as Cyclomethicones, amino-modified silicones (INCI: Amodimethicones) and silicone waxes, e.g., polysiloxane-polyalkylene copolymers (INCI: Stearyl Dimethicones and Cetyl Dimethicones) and dialkoxydimethylpolysiloxanes (Stearoxy Dimethicones), which are available as various ABIL® wax grades from Goldschmidt GmbH.

Especially when the composition is a sunscreen composition, organic sun filter substances, such as, for example, cinnamic acid derivatives; salicylic acid derivatives; camphor derivatives; triazine derivatives; benzophenone derivatives; dibenzoylmethane derivatives; diphenylacrylate derivatives; benzimidazole derivatives; p-aminobenzoic acid derivatives, polymer filters and silicone filters; more precisely:
p-aminobenzoic acid, ethoxylated p-aminobenzoate, 2-ethylhexyl p-dimethylaminobenzoate, N-propoxylated ethyl p-aminobenzoate, glyceryl p-aminobenzoate, homomenthyl salicylate, 2-ethylhexyl salicylate, triethanolamine salicylate, 4-isopropylbenzyl salicylate, 4-tert-butyl 4′-methoxydibenzoylmethane, 4-isopropyldibenzoylmethane, 2-ethylhexyl 4-methoxycinnamate, methyldiisopropyl cinnamate, isoamyl 4-methoxycinnamate, diethanolamine 4-methoxycinnamate, menthyl anthranilate, 2-ethylhexyl 2-cyano-3,3′-diphenylacrylate, ethyl 2-cyano-3,3′-diphenylacrylate, 2-, 3-(4′-trimethylammonium)benzylidene-bornan-2-one methylsulfate, urocanic acid, 2-hydroxy-4-methoxybenzophenone, 2-hydroxy-4-methoxybenzophenone-5-sulfonate, 2,4-dihydroxybenzophenone, 2,2′,4,4′-tetrahydroxybenzophenone, 2,2′-dihydroxy-4,4′-dimethoxybenzophenone, 2-hydroxy-4-n-octoxybenzophenone, 2-hydroxy-4-methoxy-4′-methylbenzophenone, 3-(4′-methylbenzylidene)-d,1-camphor, 3-benzylidene-d,1-camphor, 2,4,6-tris[p-(2′-ethylhexyl-1′-oxycarbonyl)anilino]-1,3,5-triazine, 2-[p-(tert-butylamido)anilino]-4,6-bis[(p-(2′-ethylhexyl-1′-oxycarbonyl)anilino]- 1,3,5-triazine, polymer of N-[(2 and 4)-[(2-oxoborn-3-ylidene)methyl]benzyl]acrylamide, 2-[4-(diethylamino)2-hydroxybenzoyl]alkyl benzoates, polyorganosiloxanes with malonate group, also belong to the oils and oil-like constituents for the purposes of the above definition.

Preferably, the quaternized polysaccharide is a polysaccharide selected from the group consisting of quaternized guar (“guar quat”), agar, pectin, carob seed flour, scleroglucan, xanthan, tragacanth, tamarind seed flour. Preference is given to using the guar quat and guar hydroxypropyltrimonium chloride, which is supplied, for example, by Goldschmidt GmbH under the trade name AMILAN® Guar 39. Further preferably used guar quats are, for example, the products sold under the trade names Jaguar CBS, Jaguar C-17 and Jaguar C-16, Jaguar Excel or Cosmedia Guar.

Furthermore, it is preferably provided that the polyethylene glycol derivative or polypropylene glycol derivative is a polyethylene glycol diester with a molecular weight in the range between 1 500 and 12 000 daltons. Alternatively, it may, for example, also be polypropylene glycol diesters or polyethylene/polypropylene glycol diesters.

Preferably, polyethylene glycol distearate with a molecular weight of 6000, which is supplied, for example, by Goldschmidt GmbH under the trade name REWOPAL® PEG 6000 DS, for example, is used. The latter is specified in a product information as thickener for shampoos. In this connection, concentrations of from 0.5 to 5% by weight are recommended.

Further derivatives that can be used in the above sense include, for example, PEG- 150 dibehenate, PEG-90 diisostearate, PEG-175 diisostearate, PEG-32 dilaurate, PEG-75 dilaurate, PEG-150 dilaurate, PEG-32 dioleate, PEG-75 dioleate, PEG-150 dioleate, PEG-30 dipolyhydroxystearate, PEG-20 diricinoleate, PEG-32 distearate, PEG-75 distearate, PEG-120 distearate, PEG-150 distearate, PEG-175 distearate, PEG-190 distearate, PEG-250 distearate, PEG/PPG-32/3 diricinoleate and PPG-30 dioleate.

The polymeric quaternized ammonium compound used according to the invention is one or more members of the group of cationic polymers of organically substituted quaternized ammonium compounds. These polymers are also referred to as polyquaternium. The various representatives of this group are differentiated according to the INCI nomenclature by numbers, for example polyquaternium-1, polyquaternium-42, etc. Polyquaternium compounds are often used as antistats or film formers, but also as combability aids and conditioners.

A preferably used polyquaternium is a composition which is available under the term polyquaternium-37 and has the following structural formula:

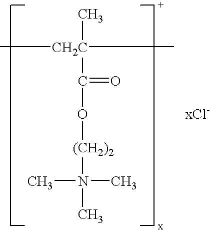

The chemical name of this substance is poly-N,N,N-trimethyl-2-((2-methyl-1-oxo-2-propenyl)oxy)chloride.

It is particularly preferably provided that the inventive composition furthermore comprises a cosmetic auxiliary and/or additive which is preferably selected from fatty substances, organic solvents, thickeners, irritation-alleviating agents, antioxidants, opacifiers, stabilizers, emollients, hydroxy acids, antifoams, hydrating agents, vitamins, perfumes, preservatives, interface-active substances, fillers, masking agents, polymers, propellants, alkalizing agents or acidifying agents, and/or colorants.

The auxiliaries or additives mentioned include, in particular, preservatives, preservation aids, bactericides, perfumes, substances for preventing foaming, dyes, pigments, which have a coloring effect, thickeners, moisturizing and/or humectant substances, fillers, which improve the skin feel, fats, oils, waxes and/or other customary cosmetic auxiliaries of a cosmetic formulation including alcohols, polyols, polymers, foam stabilizers, electrolytes, organic solvents and/or silicone derivatives. Cosmetic auxiliaries that can preferably be used according to the invention include alcohols, ethanol and/or isopropanol, diols or polyols, and ethers thereof, in particular propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilizers, electrolytes, dihydroxyacetone, and optionally one or more thickeners, such as silicon dioxide, aluminum silicates, polysaccharides, and derivatives thereof, hyaluronic acid, xanthan gum, hydroxypropylmethylcellulose, polyacrylates, carbopols, for example carbopol grades 980, 981, 1382, 2984, 5984, in each case individually or in combination.

Suitable cosmetic auxiliaries that can be used are described, for example, in EP-A-1 566 170, to which reference is made here in its entirety.

The cosmetic composition of the present invention can also have moisturizers. Moisturizers is the term used to refer to substances or substance mixtures which give cosmetic compositions the property, following application and spreading on the surface of the skin, of reducing moisture loss from the horny layer and/or of positively influencing the hydration of the horny layer. Moisturizers that can be used include glycerol, lactic acid and/or lactates, in particular sodium lactate, butylene glycol, propylene glycol, biosaccharides, glycine, soya, ethylhexyloxyglycerol, pyrrolidonecarboxylic acid and urea, and also polymeric moisturizers from the group of water-soluble and/or water-swellable and/or water-gellable polysaccharides. Also suitable are hyaluronic acid, chitosan and/or a fucose-rich polysaccharide, which is listed in the Chemical Abstracts under the registry number 178463-23-135 and is available, for example, under the name Fucogel 1000 from SOLABIA S.A.

Preservatives which can be used for the purposes of the present invention include formaldehyde donors, iodopropynyl butylcarbamates, paraben, p-hydroxybenzoic acid alkyl esters, such as methyl-, ethyl- propyl- and/or butylparaben, phenoxyethanol, ethanol, benzoic acid. Furthermore, according to the invention, the preservative system advantageously comprises preservation aids, such as, for example, octoxyglycerol, glycine and/or soya.

Antioxidants that can be used for the purposes of the present invention include water-soluble antioxidants, vitamins, in particular ascorbic acid and derivatives thereof. Preferred antioxidants are also vitamin E and derivatives thereof, and vitamin A and derivatives thereof. The amount of antioxidants, based on the total weight of the cosmetic composition, can constitute 0.001 to 30% by weight, preferably 0.05 to 15% by weight, in particular 0.5 to 10% by weight, based on the total weight of the composition.

Natural active ingredients and/or derivatives thereof that can be used include alpha-lipoic acid, phytoene, D-biotin, coenzyme Q10, alpha-glucosylrutin, carnitine, carnosine, natural and/or synthetic isoflavonoids, creatine, taurine and/or β-alanine. Added to this are secondary plant substances with antioxidative properties, in particular polyphenols and derivatives thereof.

The cosmetic composition can also comprise fillers which further improve the sensory and cosmetic properties of the formulations and, for example, bring about or enhance a velvety or silky skin feel. Fillers that can be used include starch and starch derivatives, such as tapioca starch, distarch phosphate, aluminum or sodium starch octenylsuccinate, and also pigments which have neither primarily UV filter effect nor a coloring effect, for example boron nitride and/or Aerosils (CAS No. 7631-86-9).

Furthermore, it may optionally be advantageous to incorporate film formers into the cosmetic compositions according to the invention, for example in order to improve the water resistance of the compositions or to increase the UV protection. Of suitability are both water-soluble, dispersible and/or fat-soluble film formers, in each case individually or in combination with one another. Film formers that can be used according to the invention include polyurethanes, polyisobutenes, hydrogenated polyisobutenes, dimethicone copolyol, polyacrylates, PVP/VA copolymers (PVP polyvinylpyrrolidone, VA=vinyl acetate).

Fat-soluble film formers can be selected from the group of polymers based on polyvinylpyrrolidone (PVP), copolymers of polyvinylpyrrolidone, PVP/hexadecene copolymer and/or the PVP/eicosene copolymer.

Furthermore, the cosmetic composition according to the invention can also comprise one or more supplementary hydrophilic or lipophilic organic filters that are effective in the UV-A and/or UV-B region, and/or pigments or nanopigments of metal oxides which are coated or uncoated. These may, for example, be octocrylene, octyl methoxycinnamate or butylmethoxydibenzoylmethane. Further oil-soluble UVB and/or broadband filter substances for the purposes of the present invention include 3-benzylidenecamphor derivatives, preferably 3-(4-methylbenzylidene)camphor, 3-benzylidenecamphor-4-13-aminobenzoic acid derivatives, preferably 2-ethylhexyl 4-(dimethylamino)benzoate, amyl 4-dimethylaminobenzoate; derivatives of benzophenone, preferably 2-hydroxy-4-methoxybenzophenone, 2-hydroxy-4-methoxy-4′-methylbenzophenone, 2,2′-dihydroxy-4-methoxybenzophenone, and polymer-bonded UV filters, such as 3-(4-(2,2-bis-ethoxycarbonylvinyl)phenoxy)propenyl)methoxysiloxane/dimethyisiloxane copolymer.

Furthermore, the cosmetic compositions according to the invention can comprise pigments or nanopigments with particle sizes in the range from 5 to 200 nm, preferably in the range from 10 to 50 nm, which are coated or uncoated. Nanopigments of metal oxides that are suitable for use include titanium dioxide, for example amorphous or crystalline in the form of rutile and/or anatase, iron, manganese, zinc, zirconium or cerium, which all represent known UV photoprotective substances.

Conventional coatings may be aluminum oxide, silicon dioxide, aluminum stearate, trimethoxycaprylylsilanes, triethoxycaprylylsilanes, dimethicones and/or methicones. Nanopigments of metal oxides that can be used according to the invention, and which are optionally coated are described, for example, in EP-A-0 518 772 and EP-A-0 518 773. Cosmetic compositions that can be used according to the invention may have 0. 1 to 20% by weight, preferably 0.5 to 15% by weight, and preferably 0.5 to 10% by weight, of one or more of these UV filter substances. Moreover, in a preferred embodiment, it is provided according to the invention that the composition also comprises at least one agent for tanning and/or artificially tanning the skin. This may be, for example, dihydroxyacetone.

The cosmetic composition is preferably a composition for the care or the protection of the human epidermis or a sunscreen composition which is present in the form of a nonionic vesicle dispersion, an emulsion, in particular an emulsion of the oil-in-water type, a milk, a gel, a dispersion or a spray.

In this connection, body lotions, self-tanning compositions, sunscreen compositions, aftersun lotions and the like are primarily contemplated.

Furthermore provided according to the invention is a method for the topical cosmetic treatment of the skin for the purpose of achieving a care or protective effect, wherein it consists in applying to the skin an effective amount of a composition as defined above.

The present invention is illustrated more precisely by the examples discussed below. It is to be noted that the examples are only descriptive in character and are not intended to limit the invention in any form.

The components of phase A or of phase B listed in the tables below were heated separately to 70° C., stirred together, homogenized and stirred cold down to 40° C. A preservative was then added, the product was stirred cold at 30° C., optionally phase C was stirred in and then the mixture was bottled. After 3 days, the viscosity was determined. Phase A is the phase which comprises the oils and oil-like constituents.

TABLE 1

| Constituent | % by wt. | | | | |
|---|---|---|---|---|---|
| Phase A | | | | | |
| REWOQUAT ® WE 38 | 3.5 | 3.5 | 3.5 | 3.5 | 3.5 |
| TEGIN ® M | 1.5 | 1.5 | 1.5 | 1.5 | 1.5 |
| TEGO ® Alkanol 18 | 1.0 | 1.0 | 1.0 | 1.0 | 1.0 |
| Mineral oil | 1.5 | 1.5 | 1.5 | 1.5 | 1.5 |
| TEGOSOFT ® CT | 5.0 | 5.0 | 5.0 | 5.0 | 5.0 |
| ABIL ® 100 | 1.5 | 1.5 | 1.5 | 1.5 | 1.5 |
| TEGOSOFT ® P | 1.0 | 1.0 | 1.0 | 1.0 | 1.0 |
| OMC | 5.0 | 5.0 | 5.0 | 5.0 | 5.0 |
| OC | 3.0 | 3.0 | 3.0 | 3.0 | 3.0 |
| BMDM | 2.5 | 2.5 | 2.5 | 2.5 | 2.5 |
| Phase B | | | | | |
| Glycerol | 3.0 | 3.0 | 3.0 | 3.0 | 3.0 |
| Urea | 0.5 | 0.5 | 0.5 | 0.5 | 0.5 |
| Water | 69.38 | 68.88 | 68.38 | 67.88 | 66.88 |
| REWOPAL ® PEG 6000 | 0.5 | 1.0 | 1.5 | 2.0 | 3.0 |
| DS | | | | | |
| Na₂HPO₄ * 12H₂O | 0.12 | 0.12 | 0.12 | 0.12 | 0.12 |
| (20% solution) | | | | | |
| Phenonip ® | 1.0 | 1.0 | 1.0 | 1.0 | 1.0 |
| 3 days | | | | | |
| PH | 4.5 | 4.3 | 4.1 | 3.9 | 3.6 |
| Viscosity | 60 | 80 | 120 | 240 | 560 |
| (5/10)* mPas | | | | | |

*Viscosity measurement using Brookfield RVT, spindle No. 5, 10 rpm.

TABLE 2

| Constituent | % by wt. | | | | |
|---|---|---|---|---|---|
| Phase A | | | | | |
| REWOQUAT ® | 3.5 | 3.5 | 3.5 | 3.5 | 3.5 |
| WE 38 | | | | | |
| TEGIN ® M | 1.5 | 1.5 | 1.5 | 1.5 | 1.5 |
| TEGO ® Alkanol 18 | 1.0 | 1.0 | 1.0 | 1.0 | 1.0 |
| Mineral oil | 1.5 | 1.5 | 1.5 | 1.5 | 1.5 |
| TEGOSOFT ® CT | 5.0 | 5.0 | 5.0 | 5.0 | 5.0 |
| ABIL ® 100 | 1.5 | 1.5 | 1.5 | 1.5 | 1.5 |
| TEGOSOFT ® P | 1.0 | 1.0 | 1.0 | 1.0 | 1.0 |
| OMC | 5.0 | 5.0 | 5.0 | 5.0 | 5.0 |
| OC | 3.0 | 3.0 | 3.0 | 3.0 | 3.0 |
| BMDM | 2.5 | 2.5 | 2.5 | 2.5 | 2.5 |
| Phase B | | | | | |
| Glycerol | 3.0 | 3.0 | 3.0 | 3.0 | 3.0 |
| Urea | 0.5 | 0.5 | 0.5 | 0.5 | 0.5 |
| Water | 69.68 | 69.38 | 68.88 | 68.38 | 67.88 |
| AMILAN ® Guar 39 | 0.2 | 0.5 | 1.0 | 1.5 | 2.0 |
| Na₂HPO₄ * 12H₂O | 0.12 | 0.12 | 0.12 | 0.12 | 0.12 |
| (20% solution) | | | | | |
| Phenonip ® | 1.0 | 1.0 | 1.0 | 1.0 | 1.0 |
| 3 days | | | | | |
| PH | 5.8 | 5.8 | 6.1 | 6.5 | |
| Viscosity (5/10)* | 3400 | 4300 | 15400 | 39600 | n/n |
| mPas | | | | | |

*Viscosity measurement using Brookfield RVT, spindle No. 5, 10 rpm.

TABLE 3

| | |
|---|---|
| Constituent | % by wt. |

| | | | |
|---|---|---|---|
| Phase A | | | |
| REWOQUAT ® WE 38 | 3.5 | — | — |
| FS Palm 1850 H | — | 3.5 | — |
| VARISOFT ® EQ 65 | — | — | 3.5 |
| TEGO ® Alkanol 18 | 1.0 | — | — |
| TEGIN ® M | 1.5 | 1.5 | 1.5 |
| TEGOSOFT ® CR | 1.5 | 1.5 | 1.5 |
| TEGOSOFT ® TN | 5.0 | 6.0 | 6.0 |
| TEGOSOFT ® E | 1.5 | 1.5 | 1.5 |
| TEGOSOFT ® TIS | 1.0 | 1.0 | 1.0 |
| OMC | 5.0 | 5.0 | 5.0 |
| OC | 3.0 | 3.0 | 3.0 |
| BMDM | 2.5 | 2.5 | 2.5 |
| Phase B | | | |
| Glycerol | 3.0 | 3.0 | 3.0 |
| Urea | 0.5 | 0.5 | 0.5 |
| AMILAN ® Guar 39 | 0.5 | 0.5 | 0.5 |
| REWOPAL ® PEG 6000 DS | 0.2 | 0.2 | 0.2 |
| Na₂HPO₄ * 12H₂O | 1.5 | 1.5 | 1.5 |
| (20% strength solution) | | | |
| Water | 67.8 | 67.8 | 67.8 |
| Phenonip ® | 1.0 | 1.0 | 1.0 |
| 3 days | | | |
| pH | 5.1 | 4.9 | 5.2 |
| Viscosity (5/10)* mPas | 3900 | 3600 | 9600 |

*Viscosity measurement using Brookfield RVT, spindle No. 5, 10 rpm.

TABLE 4

| | |
|---|---|
| Constituent | % by wt. |

| | | |
|---|---|---|
| Phase A | | |
| VARISOFT ® TA 100 | 3.50 | 3.50 |
| TEGIN ® M | 2.00 | 2.00 |
| TEGO ® Alkanol 18 | 0.5 | 0.5 |
| TEGO ® Care 450 | 1.50 | 1.50 |
| OC | 10.00 | 10.00 |
| TEGO ® Sun TDEC 45 | 10.00 | 8.00 |
| Ethylhexyl Salicylate | | 5.00 |
| TEGOSOFT ® TN | 4.00 | 4.00 |
| Vitamin E acetate | 0.10 | 0.10 |
| Polyquaternium-37 | 0.30 | 0.30 |
| BMDM | 3.00 | 3.00 |
| Phase B | | |
| Glycerol 99.5% strength | 3.00 | 3.00 |
| Water | 61.00 | 58.00 |
| TEGO Cosmo C 100 (Creatine) | 0.10 | 0.10 |
| Allantoin powdered | 0.10 | 0.10 |
| Phase C | | |
| Symdiol 68 T | 0.70 | 0.70 |
| (1,2-hexanediol) | | |
| Perfume | 0.20 | 0.20 |
| 3 days | | |
| PH | 4.7 | 4.8 |
| Viscosity (5/10)* mPas | 10000 | 9500 |

*Viscosity measurement using Brookfield RVT, spindle No. 5, 10 rpm.

The products used are the following substances:
REWOQUAT® WE 38: Ester quat, available from Goldschmidt GmbH,
TEGIN® M: Glycerol monostearate, available from Goldschmidt GmbH,
TEGO® Alkanol 18: Stearyl alcohol, available from Goldschmidt GmbH,
TEGOSOFT® CT: Caprylic/capric triglycerides, available from Goldschmidt GmbH,
TEGO® Sun TDEC 45: highly stable dispersion of titanium dioxide, diethylhexyl carbonate and polyglyceryl-6 polyhydroxystearate, available from Goldschmidt GmbH,
TEGO® Care 450: Polyglyceryl-3 methylglucose distearate, available from Goldschmidt GmbH,
ABIL® 100: Dimethicones, available from Goldschmidt GmbH,
TEGOSOFT® P: Isopropyl palmitate, available from Goldschmidt GmbH,
OMC: Ethylhexyl methoxycinnamate,
OC: Octocrylene,
BMDM: Butylmethoxydibenzoylmethane,
REWOPAL® PEG 6000 DS:PEG-150 distearates, available from Goldschmidt GmbH,
Phenonip: Preservative, comprising phenoxyethanol, methylparaben, butylparaben, ethyl-paraben and propylparaben, available from Clariant UK Ltd,
AMILAN® Guar 39: quaternized guar, available from Goldschmidt GmbH,
VARISOFT® EQ 65: Ester quat of methyldiethanolamines with stearic acid and 35% cetearyl alcohol, available from Goldschmidt GmbH,
TEGOSOFT® TIS: Triisostearin, available from Goldschmidt GmbH,
TEGOSOFT® E: PPG-15 stearyl ether, available from Goldschmidt GmbH,
TEGOSOFT® TN: C₁₂₋₁₅-Alkyl benzoate, available from Goldschmidt GmbH,
TEGOSOFT® CR: Cetyl ricinoleate, available from Goldschmidt GmbH,
FS Palm 1850 H: Dipalmoylethyl ester quat and stearyl alcohol,
VARISOFT® TA 100 Distearyldimethylammonium chloride, available from Goldschmidt GmbH,
Polyquaternium-37: Poly-N,N,N-trimethyl-2-((2-methyl-1-oxo-2-propenyl)oxy)chloride,
TEGO® Cosmo C 100: Creatine, available from Goldschmidt GmbH.

The product names denoted in the table with ® are registered trade marks.

Table 1 shows compositions which, besides the ester quat REWOQUAT® WE 38, comprise polyethylene glycol distearate in various concentrations. Table 2 shows compositions which, besides the ester quat REWOQUAT® WE 38, comprise the quaternized polysaccharide AMILAN® Guar 39 in various concentrations. In both cases, the constituents mineral oil, TEGOSOFT® CT, ABIL® 100 and TEGOSOFT® P, OMC, OC and BMDM form the oil or oil-like fraction, the total proportion of which is 16% by weight.

It can be clearly seen that when using 0.5 to 3% by weight polyethylene glycol distearate, very low viscosities were established which are within the claimed range. This was surprising since polyethylene glycol distearate actually functions as a thickener. Nevertheless, compared with a comparable composition without polyethylene glycol distearate, a significant improvement in stability was achieved.

It can also be seen that when using 0.2 or 0.5% by weight of AMILAN® Guar 39, an initial viscosity of 4300 mPas is established. This finding was also surprising since AMILAN® Guar 39 also functions as a thickener. Nevertheless, compared with a comparable composition without AMILAN® Guar 39, a significant improvement in stability was achieved. Higher concentrations outside of the claimed range, on the other hand, lead to viscosities outside of the claimed viscosity range.

Table 3 shows compositions which, besides ester quats (REWOQUAT® E 38 or VARISOFT® EQ 65), comprise both polyethylene glycol distearate and also AMILAN® Guar 39. Here too, stable cationic emulsions in the required viscosity range were obtained.

Table 4 shows compositions which, besides the dialkyl quat VARISOFT® TA 100, comprise polyquaternium-37. Here too, it can be seen that stable cationic emulsions in the required viscosity range were obtained.

While the present invention has been particularly shown and described with respect to preferred embodiments thereof, it will be understood by those skilled in the art that the foregoing and other changes in forms and details may be made without departing from the spirit and scope of the present invention. It is therefore intended that the present invention not be limited to the exact forms and details described and illustrated, but fall within the scope of the appended claims.

What is claimed is:

1. A cosmetic composition for application to the skin, comprising:
a) 0.05 to 0.5% by weight of a quaternized polysaccharide, 0.05 to 5.0% by weight of a polyethylene glycol derivative or polypropylene glycol derivative and/or 0.05 to 3% by weight of a polymeric quaternized ammonium compound,
b) 10 to 40% by weight of oils and/or oil-like constituents, and
c) 0.2 to 15% by weight of an ester quat and/or of a dialkyl quat.

2. The cosmetic composition as claimed in claim 1, wherein the composition has a viscosity of from 50 to 12 000 mPas at room temperature.

3. The cosmetic composition as claimed in claim 1, wherein the quaternized polysaccharide is a polysaccharide selected from the group consisting of quaternized guar (“guar quat”), agar, pectin, carob seed flour, scleroglucan, xanthan, tragacanth, and tamarind seed flour.

4. The cosmetic composition as claimed in claim 1, wherein the polyethylene glycol derivative or polypropylene glycol derivative is a polyethylene glycol diester with a molecular weight in the range between 1500 and 12 000 daltons.

5. The cosmetic composition as claimed in claim 1, further comprising at least one of a cosmetic auxiliary and/or additive which is selected from the group of fatty substances, organic solvents, thickeners, irritation-alleviating agents, antioxidants, opacifiers, stabilizers, emollients, hydroxy acids, antifoams, hydrating agents, vitamins, perfumes, preservatives, interface-active substances, fillers, masking agents, polymers, propellants, alkalizing agents or acidifying agents and colorants.

6. The cosmetic composition as claimed in claim 1, further comprising one or more supplementary hydrophilic or lipophilic organic filters that are effective in the UV-A and/or UV-B region, and/or pigments or nanopigments of metal oxides which are coated or uncoated.

7. The cosmetic composition as claimed in claim 1, further comprising at least one agent for tanning and/or artificially tanning the skin.

8. The cosmetic composition as claimed in claim 1, wherein said composition is present in the form of a nonionic vesicle dispersion, an emulsion, a milk, a gel, a dispersion or a spray.

9. A method for the topical cosmetic treatment of the skin for the purpose of achieving a care or protective effect, comprising:
applying to the skin an effective amount of a composition including:
a) 0.05 to 0.5% by weight of a quaternized polysaccharide, 0.05 to 5.0% by weight of a polyethylene glycol derivative or polypropylene glycol derivative and/or 0.05 to 3% by weight of a polymeric quaternized ammonium compound,
b) 10 to 40% by weight of oils and/or oil-like constituents, and
c) 0.2 to 15% by weight of an ester quat and/or of a dialkyl quat.

10. The method as claimed in claim 9, wherein the composition has a viscosity of from 50 to 12 000 mPas at room temperature.

11. The method as claimed in claim 9, wherein the quaternized polysaccharide is a polysaccharide selected from the group consisting of quaternized guar (“guar quat”), agar, pectin, carob seed flour, scleroglucan, xanthan, tragacanth, and tamarind seed flour.

12. The method as claimed in claim 9, wherein the polyethylene glycol derivative or polypropylene glycol derivative is a polyethylene glycol diester with a molecular weight in the range between 1500 and 12 000 daltons.

13. The method as claimed in claim 9, wherein said composition further comprises at least one of a cosmetic auxiliary and/or additive which is selected from the group of fatty substances, organic solvents, thickeners, irritation alleviating agents, antioxidants, opacifiers, stabilizers, emollients, hydroxy acids, antifoams, hydrating agents, vitamins, perfumes, preservatives, interface-active substances, fillers, masking agents, polymers, propellants, alkalizing agents or acidifying agents and colorants.

14. The method as claimed in claim 9, wherein said composition further comprises one or more supplementary hydrophilic or lipophilic organic filters that are effective in the UV-A and/or UV-B region, and/or pigments or nanopigments of metal oxides which are coated or uncoated.

15. The method as claimed in claim 9, wherein said composition further comprises at least one agent for tanning and/or artificially tanning the skin.

16. The method as claimed in claim 9, wherein said composition is present in the form of a nonionic vesicle dispersion, an emulsion, a milk, a gel, a dispersion or a spray.
